(19) United States
(12) Patent Application Publication
(10) Pub. No.: US 2017/0355658 A1
(43) Pub. Date: Dec. 14, 2017

(54) CO2-MEDIATED ETHERIFICATION OF BIO-BASED DIOLS

(71) Applicant: Archer Daniels Midland Company, Decatur, IL (US)

(72) Inventors: Chi Cheng Ma, Forsyth, IL (US); Kenneth Stensrud, Decatur, IL (US); Padmesh Venkitasubramanian, Forsyth, IL (US)

(21) Appl. No.: 15/535,289

(22) PCT Filed: Nov. 19, 2015
(86) PCT No.: PCT/US15/61559

§ 371 Date: Jun. 12, 2017

Related U.S. Application Data
(60) Provisional application No. 62/093,730, filed on Dec. 18, 2014.

(51) Int. Cl. C07C 68/06; C07C 68/04; C07C 41/16; C07D 493/04; C07D 307/42; C07D 307/12; C07C 43/10; C07C 43/12

(52) U.S. Cl. CPC C07C 68/06; C07D 307/42; C07C 68/04; C07D 307/12; C07C 41/16; C07D 493/04; C07C 43/12; C07C 43/10

(57) ABSTRACT

A method of etherifying glycols or other diols by employing renewable reagents is disclosed. In particular, the method involves contacting a diol with an alkylating agent in an alcoholic solvent, catalyzed with a catalyst (carbonic acid) generated in situ (from CO₂). The mono- and di-ether products can serve as valued precursors to an array of renewable surfactants, dispersants, and lubricants, among others.

### CLAIM OF PRIORITY

This application claims benefit of priority from U.S. Provisional Application No. 62/093,730, filed Dec. 18, 2014, the contents of which are incorporated herein by reference.

FIELD OF INVENTION

The present disclosure relates to a process for selective etherification of polyols using renewable reagents. In particular, the process involves generating mono- and di-alkyl ethers of bio-based glycols and other diols.

### BACKGROUND

Glycol ethers are used in various industrial applications as components of solvents, coatings, inks, and household cleaners. The current convention for large industrial-scale preparation of glycol ethers arose from petroleum-based olefin epoxidization, which is followed by catalytic solvolysis with an alcohol to generate mono- and diether products that are separated by means of fractional distillation. However, interest in “green” renewable resources has in recent years spurred an effort to develop an alternative, more sustainable means to supply the large volume demand for glycol ethers.

Present conventional processes for converting glycols to ethers involves a reaction with an alcohol using a strong acid catalyst, such as sulfuric acid (H₂SO₄). The reaction generates desired ethers from the glycol as well as side products, such as dimethyl ether, which can be explosive. Industry usually burns off this side product, which contributes unfortunately additional atmospheric CO₂.

By virtue of its nature as the principal product from fossil fuel combustion and imputed culpability as a climate changing “greenhouse gas,” CO₂ has attracted much media attention as a byproduct to be reduced. Efforts to curb CO₂ emissions through various regulatory measures have been marked with limited success, in part, owing to the rapid growth of the economies of some developing nations that are sharply driven by abundant, energy rich oil and coal. Sequestration or capture and storage of CO₂ in deep underground reservoirs affords a temporary solution for containment of increasing atmospheric CO₂ levels. However, several drawbacks exist, including a need for highly toxic chemicals with potential for widespread groundwater contamination, and sometimes uncertain long term seismic effects.

Another branch of CO₂ research focuses on the capture and utilization of the gas either as a one carbon additive (C1 unit). Finally, an emerging interest is in using pressurized CO₂ to catalyze processes in aqueous solutions (carbonic acid catalysts). This area holds tremendous potential for several reasons, including a) the preclusion of catalyst removal (carbonic acid spontaneously decomposes to water and CO₂ upon depressurization); b) the “green” aspects of utilizing CO₂ and water as principal components driving a chemical transformation; and c) propitious process economics stemming from these bountiful, inexpensive materials. CO₂ catalyzed transformations have several precedents. For example, Shirai et al. (Green Chemistry 2009, 48-52) state that CO₂ is deployed to actuate the dehydrative cyclization of multiple polyols to the corresponding cyclic ethers in a high temperature aqueous matrix. In another example, Savage et al. (Ind. Eng. Chem. Res. 2003, 290-294), deploy CO₂ to promote the etherification ofp-cresol with t-butyl alcohol in high temperature water. In another example, Zhu and co-workers disclose a preparation of propylene glycol dimethyl ether by means of deploying dimethyl carbonate and sodium or potassium hydroxide (Faming Zhuanli Shenqing Gongkai Shuomingshu, published Chinese Patent Application No. 1554632 (15 Dec. 2004)).

In view of the foregoing needs and technical developments, a way that can leverage the capture and use of CO₂ to develop a “green” synthesis process for the generation of glycol or diol ethers would be a welcome innovation for industrial and manufacturing uses.

### SUMMARY OF THE INVENTION

The present disclosure describes in part a process for preparing mono- or dialkyl ethers. The method involves contacting a diol with an alkylating agent in an alcoholic solvent, in the presence of a catalyst that generates in situ a weak acid, at a temperature for a sufficient time to convert the diol to a corresponding alkyl ether. The diol can be at least an isohexide (i.e., isosorbide, isomannide, and isoidide), a reduction product of 5-ydroxymethylfurfural (HMF) (i.e., furan-2,5-diyldimethanol (FDM), ((2R,5S)-tetrahydrofuran-2,5-diyl)dimethanol and ((2S,5S)-tetrahydrofuran-2,5-diyl)dimethanol (bHMTHFs)), ethylene glycol (EG), propylene glycol (PG), 2,3 butane diol (BDO) or 1,6 hexane diol. The alkylating agent is an alkyl carbonate. The weak acid is carbonic acid that is formed in situ from hydrated carbon dioxide (CO₂) catalyst. The carbonic acid disappears after depressurization of the reaction.

In another aspect, the present disclosure also describes a process for making polyethers or epoxides from some of the mono- or dialkyl ethers prepared according to the foregoing process above. This second process involves reacting a diol with an alkylating agent in an alcoholic solvent, catalyzed by a weak acid generated in situ, generating an allyl ether, and at least polymerizing or epoxidizing the allyl ether. Like in the underlying etherification process, the mono- or dialkyl ethers are derived from a diol selected from the group consisting of ethylene glycol (EG), propylene glycol (PG), 2,3 butane diol (BDO), 1,6 hexane diol, isosorbide, isomannide, isoidide, furan-2,5-diyldimethanol (FDM), ((2R,5S)-tetrahydrofuran-2,5-diyl)dimethanol and ((2S,5S)-tetrahydrofuran-2,5-diyl)dimethanol (bHMTHFs), and can serve as valued precursors or renewable feedstocks for various industrial applications.

Additional features and advantages of the present process will be disclosed in the following detailed description. It is understood that both the foregoing summary and the following detailed description and examples are merely representative of the invention, and are intended to provide an overview for understanding the invention as claimed.

### BRIEF DESCRIPTION OF FIGURES

FIGS. 1A and 1B, respectively, are gas chromatographic/mass spectroscopic (GC/MS) chromatogram and mass spectrum of FDM dissolved in methanol (MeOH).

FIG. 2 is a mass spectrum of product generated according to an embodiment of the present process showing that most of the FDM has converted to its monoether analog and a significant amount of diether analog.

FIGS. 3A, 3B, and 3C, respectively, are a GC chromatogram and two mass spectra of another embodiment using FDM, which manifests a similar product profile as in the embodiment of FIG. 2. In the GC/MS chromatogram the attenuated FDM signal (10.999 min.) demonstrates greater conversion, and the more intense signals for the diether analog (9.765 min.) and monoether analog (10.337 min.) indicate greater conversion to those species.

FIG. 4 is a GC/MS chromatogram of a comparative reaction in which CO₂ was absent or present in an insufficient amount, showing a single, prominent peak that consists of unreacted FDM and a lesser peak at 10.334 min. corresponding to a monoether analog.

FIG. 5 is a GC/MS chromatogram of a comparative example in which the alcohol solvent (MeOH) was absent or present in an insufficient amount, showing a single signal of unreacted FDM at 11.044 min.

FIG. 6A is a GC/MS chromatogram of a comparative example in which the alkyl carbonate (DMC) was absent or present in an insufficient amount, showing the conversion of FDM to monoether and diether analogs.

FIGS. 6B and 6C, respectively, are mass spectra for the monoether and diether analogs from FIG. 6A.

FIG. 7A is a GC/MS chromatogram of products according to an embodiment using bHMTHF, showing the cis diether analog (13.8) as a major product and trans diether analog (14.0) as a minor product.

FIGS. 7B and 7C, respectively, are mass spectra for the cis dimethyl ether and trans dimethyl ether analogs.

FIG. 8, is a GC/MS chromatogram of a comparative example in which CO₂ was absent or present in an insufficient quantity, showing unreacted bHMTHF in MeOH, with a 9:1 cis:trans diastereomer ratio.

FIG. 9, is a GC/MS chromatogram of a comparative example in which the alcohol solvent (MeOH) was absent or present in an insufficient quantity, showing unreacted bHMTHF and virtually no ether products.

FIG. 10, is a GC/MS chromatogram of a comparative example in which the alkyl carbonate (DMC) was absent or present in an insufficient quantity, showing unreacted bHMTHF and virtually no ether products.

FIGS. 11A and 11B, respectively, are a GC/MS chromatogram and mass spectrum showing propylene glycol (PG) in MeOH as starting material.

FIG. 12A, is a gas chromatogram of the isomers A and B of propylene glycol (PG)-monomethyl ether in two peaks and unreacted PG.

FIGS. 12B and 12C are two mass spectra for isomers A and B of the PG-monomethyl ether signals at 2.126 min. and 2.178 min., respectively, in the gas chromatogram detailed in FIG. 12A.

FIG. 13A, is a gas chromatogram of the mixed mono- and di-methyl ether products of PG etherification according to an embodiment of the present process.

FIGS. 13B, 13C and 13D, are mass spectra corresponding to the signals in the gas chromatogram detailed in FIG. 13A, and representing respectively unreacted PG, PG dimethyl ether (1,2-dimethoxypropane), and isomers of PG monomethyl ether (1-methoxypropan-2-ol and 2-methoxypropan-1-ol).

FIG. 14A is a gas chromatogram of isosorbide allylation products according to another embodiment.

FIG. 14B is a mass spectrum showing a signal at 11.465 min., which is unreacted isosorbide.

FIG. 14C is a mass spectrum showing a signal at 12.961 min., which is consistent with isosorbide monoallyl ether isomers.

### DETAILED DESCRIPTION OF THE INVENTION

### SECTION I.—DESCRIPTION

Glycols and other diols that are derived from plant or bio-based feedstocks embody a value-added class of compounds, which have potential and versatility in many applications that range, for example, from polymer building blocks to pre-surfactant substrates. Researchers have pursued cost-effective processes that selectively convert monosaccharides and their corresponding reduced analogs to cyclic and linear glycols (precursors with far-ranging utilities in and of themselves) or as either oxidized or reduced variants.

The present disclosure describes a process for efficiently converting bio-based diols to mono- and di-alkyl ethers deploying renewable, environmentally innocuous alkyl carbonates in an alcoholic solvent and a traceless catalyst. As used herein, the term “traceless catalyst” refers to a species that is generated in situ during a pressurized chemical reaction and dissipates after the reaction is depressurized. This etherification approach allows for high rates of conversions of diols under relatively mild conditions that have heretofore not been seen. This process is underscored by the presence of hydrated carbon dioxide, an ingredient that can serves as a source of in situ generated acid catalyst (i.e., carbonic acid), which drives the etherification.

According to an embodiment, the diol can be a cyclic dehydration derivative of a sugar alcohol, referred to herein as isohexides. The isohexide can be at least one of isosorbide, isomannide, and isoidide. In another embodiment, the diol can be furan-2,5-dimethanol (FDM), a compound made by the partial reduction of fructose-derived 5-hydroxymethylfurfural (HMF). In yet another embodiment, the diol can be ((2R,5S)-tetrahydrofuran-2,5-diyl)dimethanol and ((2S,5S)-tetrahydrofuran-2,5-diyl)dimethanol (bHMTHFs), which are reduced products engendered from the aforementioned HMF. In other embodiments, ethylene glycol (EG) or propylene glycol (PG), a glycerol-dehydrated product, is converted to its corresponding mono- and dimethyl ethers. In still other embodiments, the diol can be 2,3 butane diol (BDO) or 1,6 hexane diol.

All of these compounds can be transformed to corresponding mono- and dialkyl ethers at relatively high conversion rates of greater than 50 wt. % of the starting diol. The conversion rate can be about 60 wt. % or greater, typically about 70 wt. % or 75 wt. % to about 95 wt. % or 100 wt. %. In certain preferred embodiments, the diol is transformed to the mono- or dialkyl ethers at about 80 wt. % or 85 wt. % to about 98 wt. % or 100 wt. % yield.

The alkylating agent is an alkyl carbonate, such as, dimethyl carbonate (DMC), diethyl carbonate (DEC), or dipropyl carbonate (DPC). A significant excess of alkyl carbonate helps with the formation of the ethers. Thus, the amount of alkyl carbonate present relative to the diol reagent is in stoichiometric excess minimally by about 2× or more. In certain embodiments, the amount of alkyl carbonate can range from about 4×, 5× or 6× to about 10× or 12× greater.

The alcohol solvent is at least a primary alcohol. Examples may include an allyl alcohol, such as, methanol (MeOH), ethanol (EtOH), propanol, and butanol. Also, the amount of alcoholic solvent present is in excess minimally by about 2× or more than that of the diol reagent. Desirably in some embodiments, the alcohol solvent is present from about 4×, 5× or 6× to about 8×, 10× or 12× greater.

In certain embodiments, the alkyl carbonate and alcohol solvent can be either the same or different alkane R-group species. However, preferably they are the same alkane R group.

An illustrative reaction of the present process according to an embodiment is delineated in Scheme A, which shows glycol methyl etherification with dimethylcarbonate in CO₂ saturated methanol (PG example).

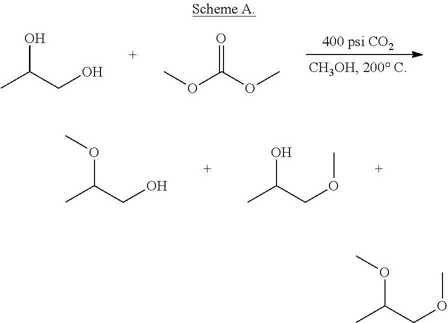

An attractive characteristic of dimethylcarbonate (DMC) is the fact that it is non-toxic and gives rise only to CO₂ and methanol which are recoverable as the byproducts. DMC has gained prominence as a “green” reagent in either acid- or base-catalyzed methylation or methoxycarbonylation of anilines, phenols, active methylene compounds and carboxylic acids. The present etherification using an alkyl carbonate like DMC is a new pathway to more versatile uses of bio-based diols. For instance, in certain embodiments, for example, the alkyl ethers of FDM or bHMTHFs can be easily converted in a subsequent oxidation step to their corresponding mono- or diesters.

Particular to the present process for attaining a high degree of glycol conversion to complementary ethers is the combination of three components (i.e., carbon dioxide, alkyl carbonate, and alcohol solvent) in the reaction. It is believed that an interplay of a CO₂ atmosphere, organo-carbonate and hydroxyl solvent enhances the formation of ethers for the diols. The CO₂ in the atmosphere during the reaction forms carbonic acid in the presence of water. As demonstrated in the comparative examples of the Examples section below, an absence or insufficient quantity of any one of these three components will result in either negligible or no conversion of the diols into their corresponding ethers. Even more, no ether products are produced from the diols when at least two of the three components are absent or present in insufficient quantities.

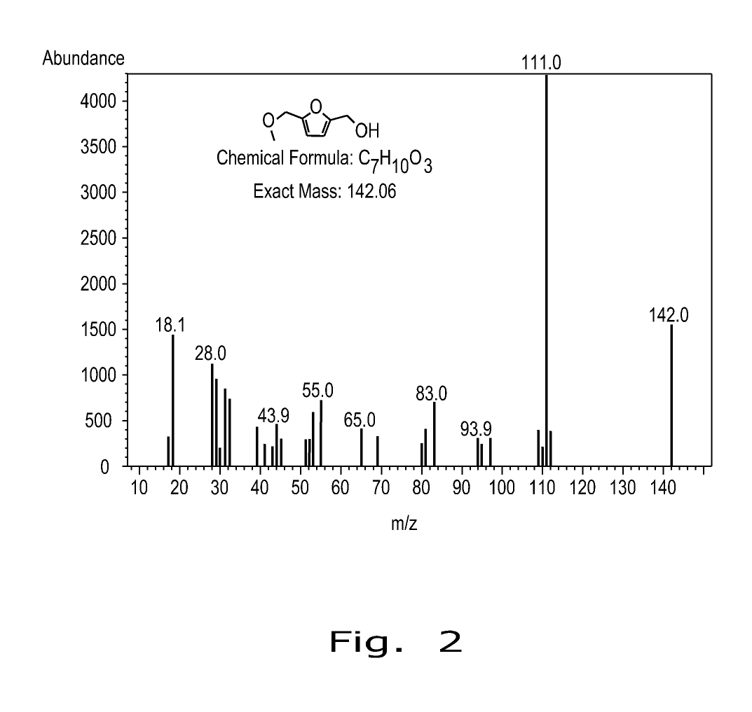

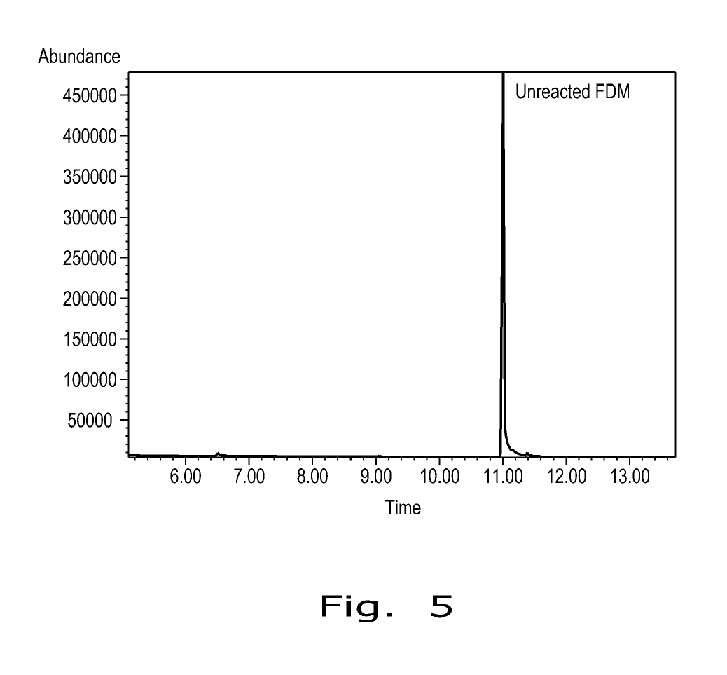

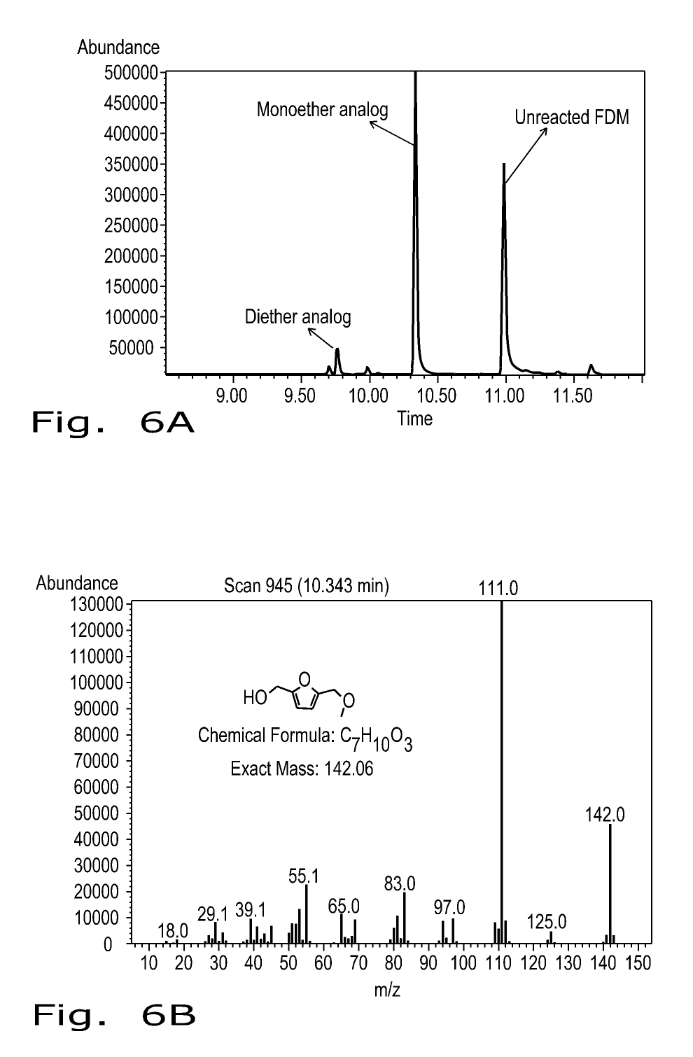

For instance, FIG. 3A and FIG. 6A, respectively, are GC chromatograms that summarize the the results of Example 2 and Comparative Example 3, which both involve etherification of FDM according to the present processes. In Example 2 all three components—CO₂, alcohol solvent, alkyl carbonate—were present in sufficient quantities. In Comparative Example 2 the alkyl carbonate was either absent or not present in sufficient quantities. A comparison of the GC chromatograms show that significant amount of unreacted FDM remain, even though the reaction generated small amounts of mono- and diether products from the FDM in Comparative Example 3. In contrast, the reaction of Example 2 has significantly less unreacted FDM and generated more of both mono- and diethers. The difference in the amount of product and unreacted starting materials, we believe is due to a synergistic effect of an interaction of the combined components. In another illustration, in Comparative Examples 1, 4, and 7, involving FDM, bHMTHFs, and PG respectively, the reactions performed without the presence of CO₂ generated no ether products.

The present etherification is conducted in an enriched CO₂ environment. That is, the reaction is performed in an atmosphere having at least 5% CO₂, and preferably about 50% CO₂ or greater. The CO₂ atmosphere can be at an initial pressure before heating of about 100 psi or 200 psi. Generally, CO₂ pressures for satisfactory glycol conversion are at about 400 psi prior to heating and about 2000 psi once the desired reaction temperature is attained. In some embodiments, the CO₂ can be at an initial pressure of about 700 psi or 800 psi. Lower initial CO₂ pressures of about 100 psi or 200 psi (1000 psi at reaction temperature) appears adequate to induce carbonic acid catalysis of the etherification process. Pressures over 1000 psi (˜4000 psi at reaction temperature) appear not to further enhance the process kinetics.

The reaction temperature can be at about 150° C., with some embodiments at about 250° C. or 260° C. The typical temperature for the reaction is about 200° C. to about 230° C., which affords satisfactory etherification of the glycols with mitigated side product formation. Reactions conducted at lower temperatures from 150° C. to 190° C. or 195° C. generated fewer side products but showed lower yields relative to reactions at higher temperatures. Reactions at higher temperatures around 260° C. or more furnished greater ether yields but tended also to manifest greater concentrations of unidentified side products, which can impede facile product isolation that is another advantage of the present process.

The reaction can be conducted for a duration of several hours, for instance from about 3 hours to about 8 or 12 hours. Typically, the reaction time is about 5 or 6 hours. One anticipates that mono- and diether yields are proportionate to the duration of the reaction; negligible at shorter time intervals, and greater enhanced amounts at longer intervals.

### SECTION II.—EXAMPLES

The following examples and accompanying gas chromatograms and mass spectra present some of the ether products that are generated according to the present processes. In “controls” or comparative examples where one or more of the reagent species is either missing or present in insufficient quantities, the data illustrate the tri-component (i.e., alcohol, alkyl carbonate, and CO₂) nature of the reactions. In other words, when a component reagent is either absent or not in proper proportion, the reaction will tend to not attain satisfactory yields of ether products.

### A. Mono- and Di-Methyl Ethers of Furan-2,5-diyldimethanol (FDM)

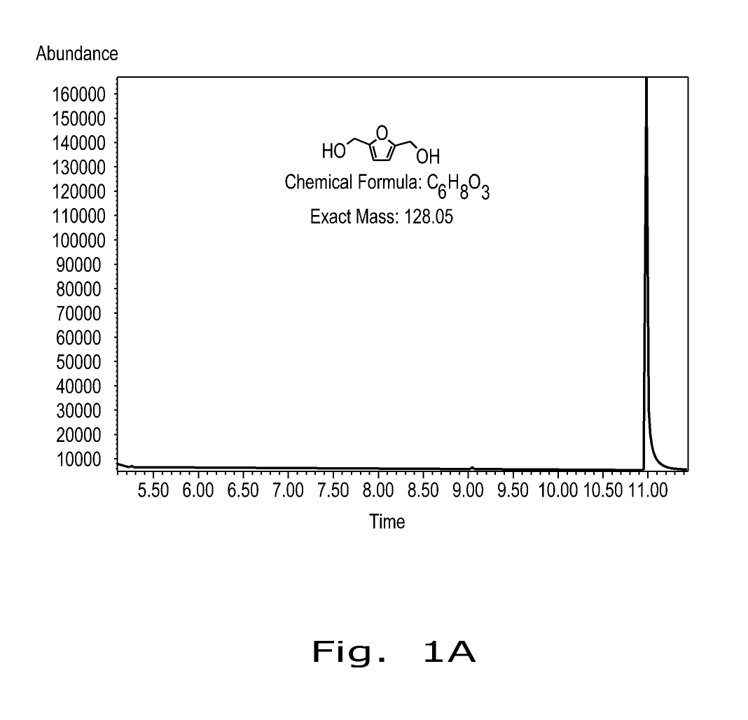

As a basis for comparison, FIGS. 1A and 1B, respectively, shows the gas chromatogram (GC) and mass spectrum of FDM dissolved in methanol (MeOH) as a baseline standard for the starting material.

Scheme 1 shows the etherification of FDM according to an embodiment described in Example 1.

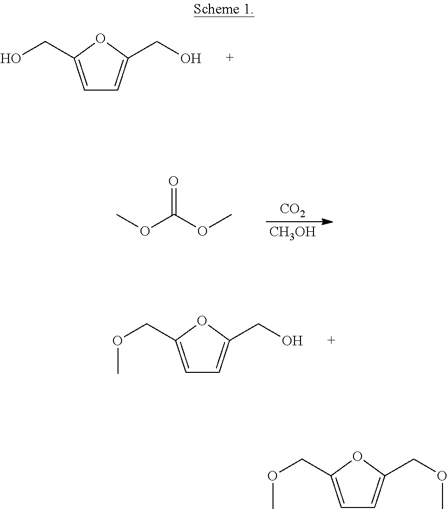

### Example 1

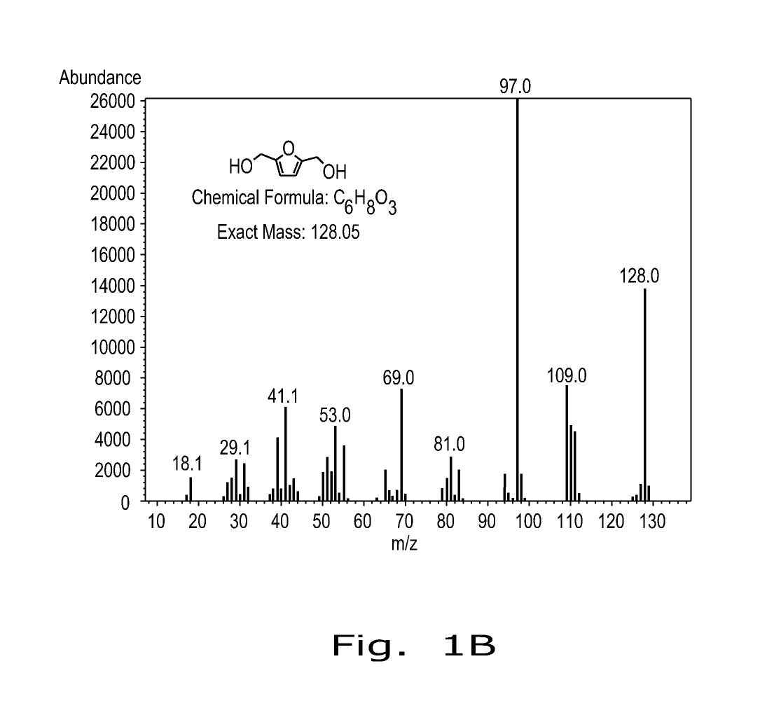

Experimental conditions (MeOH, DMC, CO₂, 3 h). A 250 cc Hastelloy pressure vessel was charged with 10 g of 2,5-furandimethanol (FDM, 78 mmol), 50 g of dimethyl carbonate (DMC, 555 mmol, 7.11 eq.) and 50 g of MeOH. The vessel was then sealed tightly and affixed to the reactor apparatus, purged ×3 with 400 psi of CO₂, then saturated with CO₂ until the pressure remained steady at 300 psi (methanol absorbs considerably amounts of CO₂). While stirring at 700 rpm, the vessel was heated to 200° C., where the reaction persisted for 3 h; the maximum pressure attained was 1650 psi at this temperature. After that time, the solution was cooled to ambient temperature, gas released, and stirring halted. The resultant brownish solution was then analyzed by GC/MS (70° C. initial temp, hold for 4 min, then 10° C. per minute until 300° C., hold for 10 min), which indicated that most of the FDM had been converted to monoether analog and a significant amount of the diether analog as shown in FIG. 2.

### Example 2

Experimental conditions (MeOH, DMC, CO₂, 5 h). A 250 cc Hastelloy pressure vessel was charged with 10 g of 2,5-furandimethanol (FDM, 78 mmol), 50 g of dimethyl carbonate (DMC, 555 mmol, 7.11 eq.) and 50 g of methanol. The vessel was then sealed tightly and affixed to the reactor apparatus, purged ×3 with 400 psi of CO₂, then saturated with CO₂ until the pressure remained steady at 400 psi (methanol absorbs considerable amounts of CO₂). While stirring at 700 rpm, the vessel was heated to 200° C., where the reaction persists for 5 h; the maximum pressure attained is 1650 psi at this temperature. After this time, the solution was cooled to ambient temperature, gas released, and stirring halted. The resulting reddish, transparent solution was then analyzed by GC/MS, which manifest a similar product profile as in Example 1, but with an attenuated FDM signal (10.999 min) demonstrating greater conversion and more intense diether analog (9.765 min) and monoether analog (10.337 min), as shown in FIGS. 3A, 3B, and 3C.

### Comparative Example 1

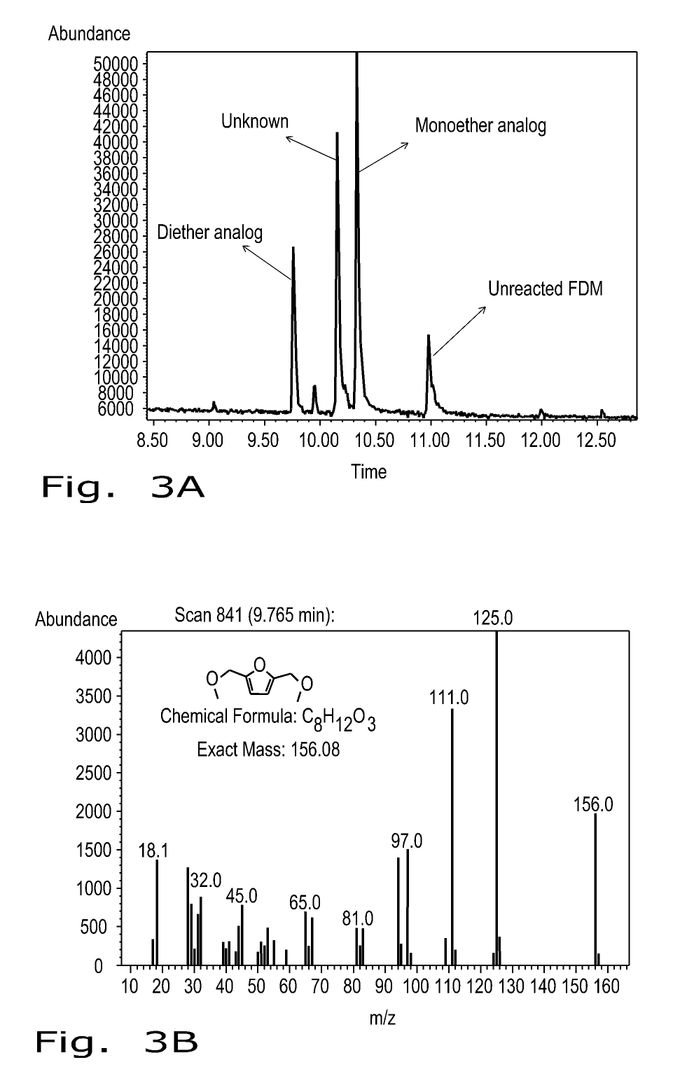

Experiment conditions (No CO₂). A 250 cc Hastelloy pressure vessel was charged with 10 g of 2,5-furandimethanol (FDM, 78 mmol), 50 g of dimethyl carbonate (DMC, 555 mmol, 7.11 eq.) and 50 g of methanol (MeOH). The vessel was then sealed tightly and affixed to the reactor apparatus, and heated to 200° C. with an overhead stirring rate of 700 rpm for 5 h. After that time, the solution was cooled to ambient temperature and stirring halted. The resultant brownish solution was then analyzed by GC/MS (70° C. initial temp, hold for 4 min, then 10° C. per minute until 300° C., hold for 10 min), disclosing a single, prominent peak that consisted of unreacted FDM and a lesser peak at 10.334 min corresponding to the monoether analog, as shown in FIG. 4.

### Comparative Example 2

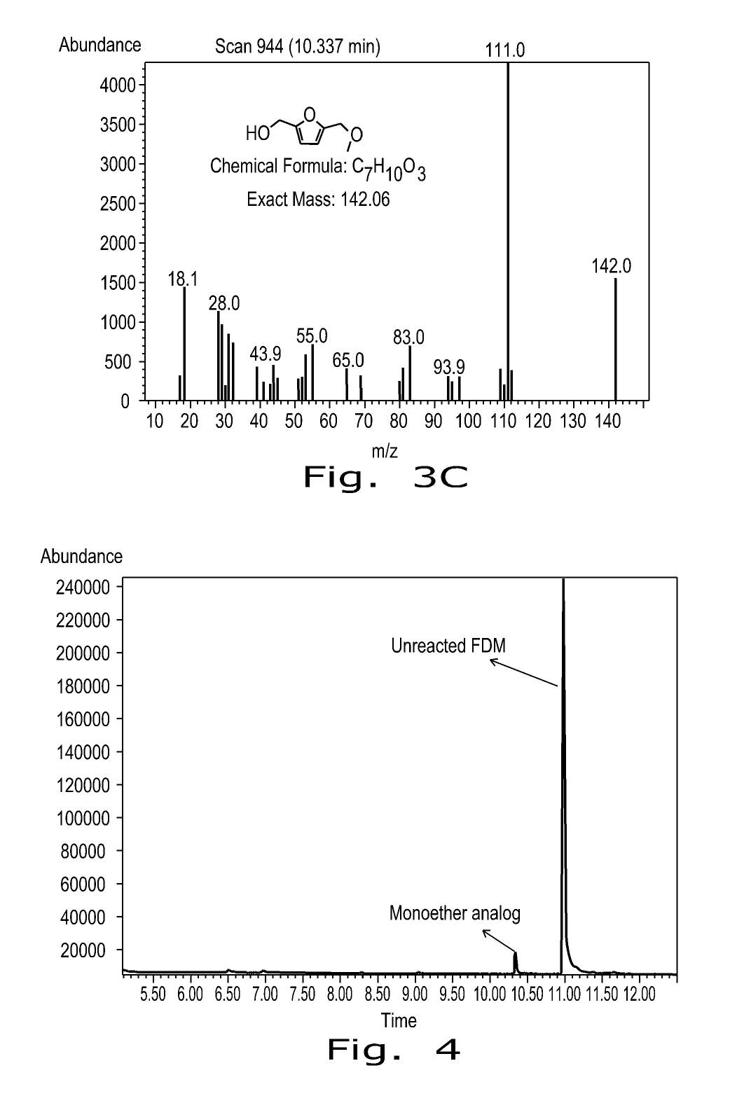

Experimental condition (No MeOH). A 250 cc Hastelloy pressure vessel was charged with 10 g of 2,5-furandimethanol (FDM, 78 mmol), 100 g of dimethyl carbonate (DMC, 1.10 mol, ˜15 eq.) and 1 g of water. The vessel was then sealed tightly and affixed to the reactor apparatus, purged ×3 with 400 psi of CO₂, then saturated with CO₂ until the pressure remained steady at 400 psi (methanol absorbs considerable amounts of CO₂). While stirring at 700 rpm, the vessel was heated to 200° C., where the reaction persists for 5 h; the maximum pressure attained is 1605 psi at this temperature. After this time, the solution was cooled to ambient temperature, gas released, and stirring halted. The resulting yellow, transparent solution was then analyzed by GC/MS using the aforementioned analytical method, exhibiting a lone signal at 11.044 min, primary to unreacted FDM, as shown in FIG. 5.

### Comparative Example 3

Experimental conditions (No DMC). A 250 cc Hastelloy pressure vessel was charged with 10 g of 2,5-furandimethanol (FDM, 78 mmol), 100 g of MeOH and 1 g of water. The vessel was then sealed tightly and affixed to the reactor apparatus, purged ×3 with 400 psi of CO₂, then saturated with CO₂ until the pressure remained steady at 400 psi (methanol absorbs considerable amounts of CO₂). While stirring at 700 rpm, the vessel was heated to 200° C., where the reaction persists for 5 h; the maximum pressure attained is 1605 psi at this temperature. After this time, the solution was cooled to ambient temperature, gas released, and stirring halted. The resulting reddish, transparent solution was then analyzed by GC/MS, using the aforementioned analytical method, and revealing three salient signals at 10.998 min (residual FDM) and 10.3343 (monoether) and 9.764 min (diether), as shown in FIGS. 6A, 6B, and 6C.

### B. Mono- and Di-Methyl Ethers of ((2R,5S)-tetrahydrofuran-2,5-diyedimethanol and Diastereomer (bHMTHFs)

### Scheme 2 shows an embodiment of bHMTHF etherification according to Example 3.

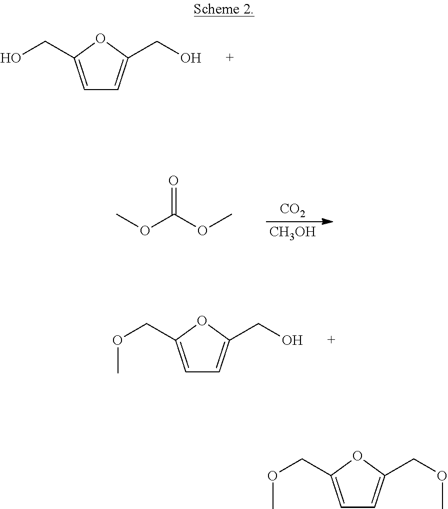

### Example 3.

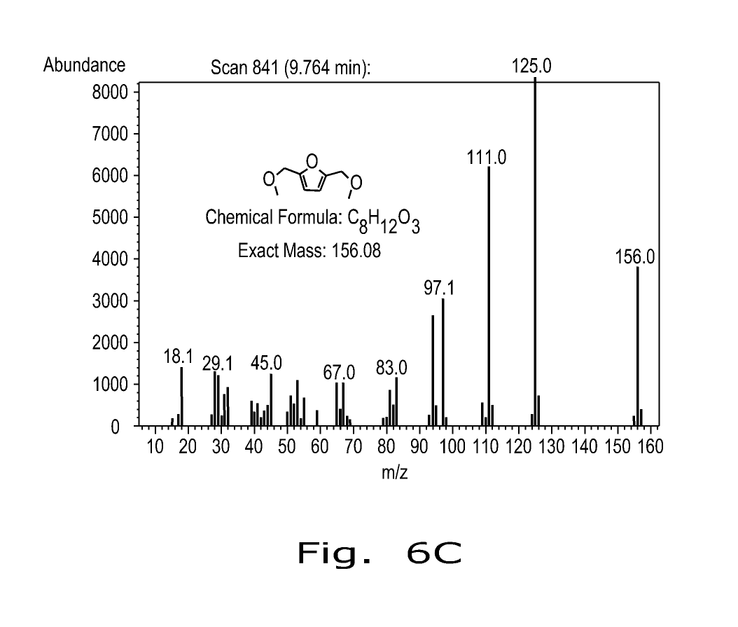

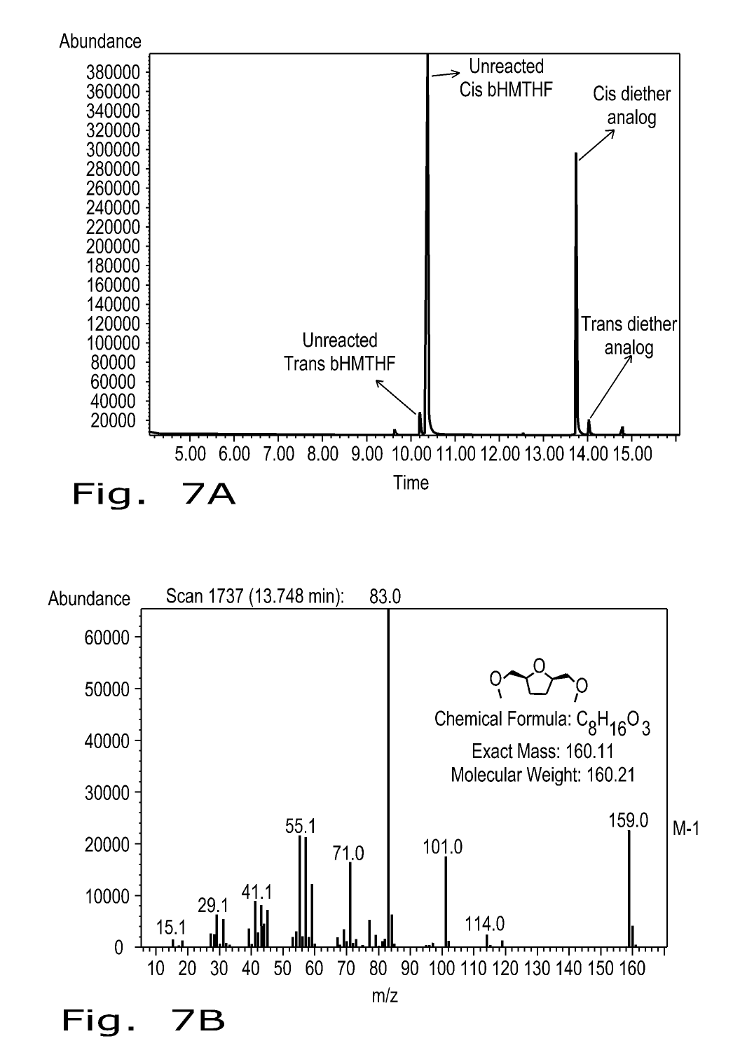

Experimental condition (MeOH, DMC, CO₂). A 250 cc Hastelloy pressure vessel was charged with 10 g of bHMTHFs (76 mmol), 50 g of dimethyl carbonate (DMC, 555 mmol, 7.11 eq.) and 50 g of methanol. The vessel was then sealed tightly and affixed to the reactor apparatus, purged ×3 with 400 psi of CO₂, then saturated with CO₂ until the pressure remained steady at 400 psi (methanol absorbs considerably amounts of CO₂). While stirring at 700 rpm, the vessel was heated to 200° C., where the reaction persisted for 5 h; the maximum pressure attained was 1740 psi at this temperature. After that time, the solution was cooled to ambient temperature, gas released, and stirring halted. The resultant brownish solution was then analyzed by GC/MS (70° C. initial temp, hold for 4 min, then 10° C. per minute until 300° C., hold for 10 min), which manifest two sets of salient peaks; a) the first set at 10.21 and 10.35 min, respectively, designated unreacted THF-diols; b) the second set at 13.88 min (cis) and 14.02 min exhibited m/z of 159.0, consistent with the target dimethoxymethyl ethers, as shown in FIG. 7A. FIGS. 7B and 7C show the mass spectrum of the cis and trans diether analogs respectively.

### Comparative Example 4

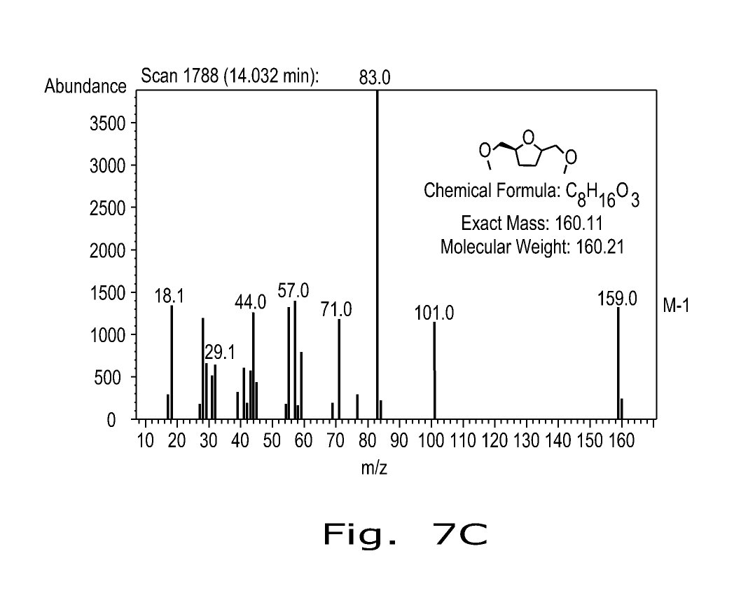

Experimental condition (No CO₂). A 250 cc Hastelloy pressure vessel was charged with 10 g of 2,5-bishydroxymethyltetrahydrofuran (THF-diols, 76 mmol), 50 g of dimethyl carbonate (DMC, 555 mmol, 7.11 eq.) and 50 g of MeOH. The vessel was then sealed tightly and affixed to the reactor apparatus, and heated to 200° C. with an overhead stirring rate of 700 rpm for 5 h. After that time, the solution was cooled to ambient temperature and stirring halted. The resultant brownish solution was then analyzed by GC/MS (70° C. initial temp, hold for 4 min, then 10° C. per minute until 300° C., hold for 10 min), disclosing a single, prominent peak and a juxtaposed, lesser peak that consisted of unreacted bHMTHFs, as presented in FIG. 8.

### Comparative Example 5

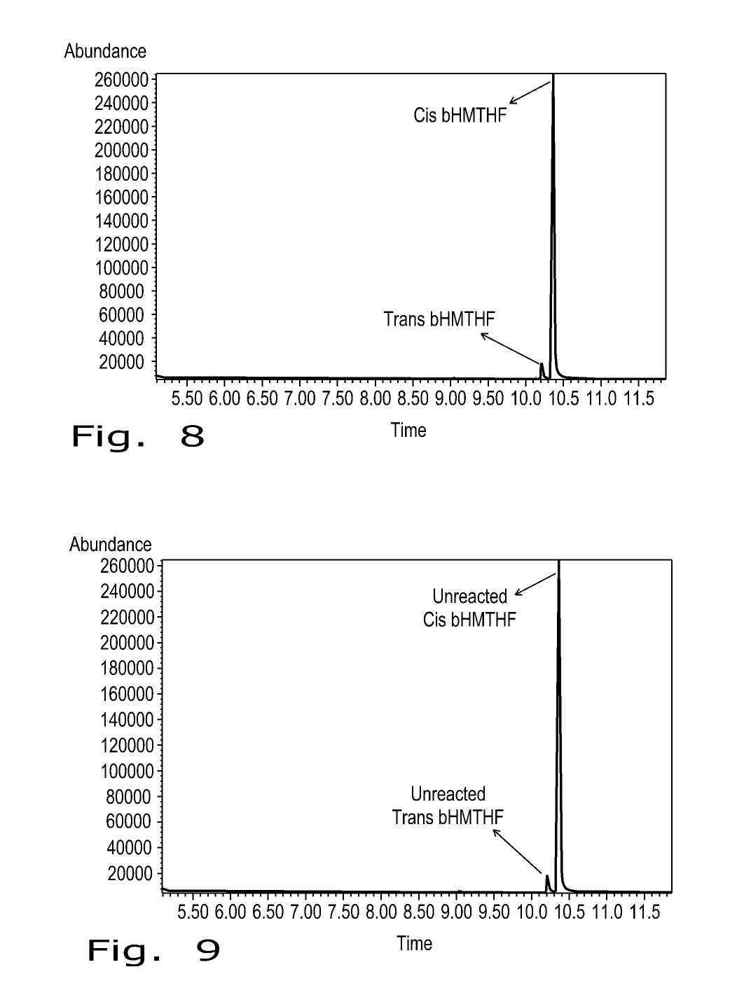

Experimental condition (No MeOH). A 250 cc Hastelloy pressure vessel was charged with 10 g of 2,5-bishydroxymethyltetrahydrofuran (THF-diols, 76 mmol), 100 g of dimethyl carbonate (DMC, 1.10 mol, ˜14.3 eq.). The vessel was then sealed tightly and affixed to the reactor apparatus, purged ×3 with 400 psi of CO₂, then saturated with CO₂ until the pressure remained steady at 400 psi (methanol absorbs considerably amounts of CO₂). While stirring at 700 rpm, the vessel was heated to 200° C., where the reaction persisted for 5 h; the maximum pressure attained was 1725 psi at this temperature. After that time, the solution was cooled to ambient temperature, gas released, and stirring halted. The resultant brownish solution was then analyzed by GC/MS (70° C. initial temp, hold for 4 min, then 10° C. per minute until 300° C., hold for 10 min) revealed salient peaks at 10.21 and 10.35 min (unreacted cis and trans bHMTHFs) as shown in FIG. 9.

### Comparative Example 6

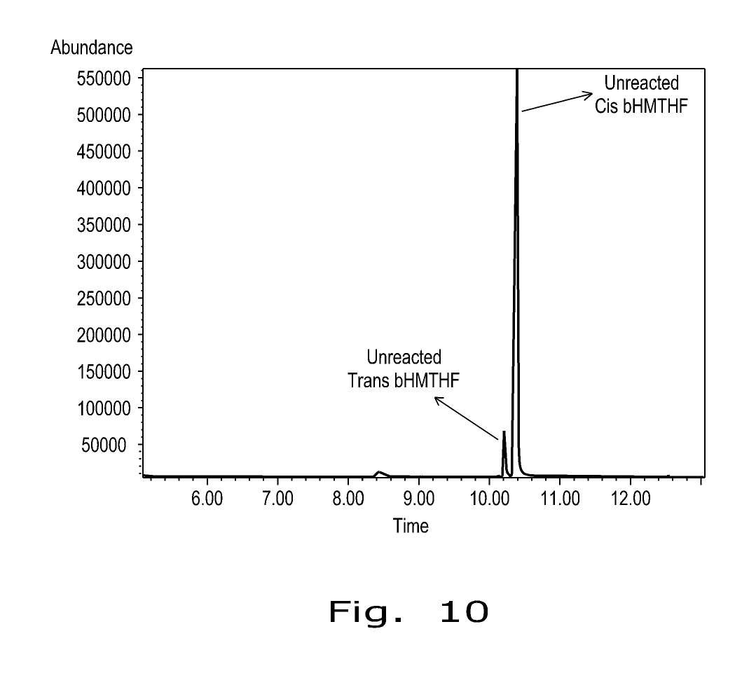

Experimental condition (No DMC). A 250 cc Hastelloy pressure vessel was charged with 10 g of 2,5-bishydroxymethyltetrahydrofuran (bHMTHFs, 76 mmol), 100 g of methanol The vessel was then sealed tightly and affixed to the reactor apparatus, purged ×3 with 400 psi of CO₂, then saturated with CO₂ until the pressure remained steady at 400 psi (methanol absorbs considerably amounts of CO₂). While stirring at 700 rpm, the vessel was heated to 200° C., where the reaction persisted for 5 h; the highest pressure attained was 1775 psi at this temperature. After that time, the solution was cooled to ambient temperature, gas released, and stirring halted. The resultant brownish solution was then analyzed by GC/MS (70° C. initial temp, hold for 4 min, then 10° C. per minute until 300° C., hold for 10 min), which disclosed only signals relating unreacted bHMTHFs, as presented in FIG. 10.

### C. Mono- and Di-Methyl Ethers of Propylene Glycol (PG)

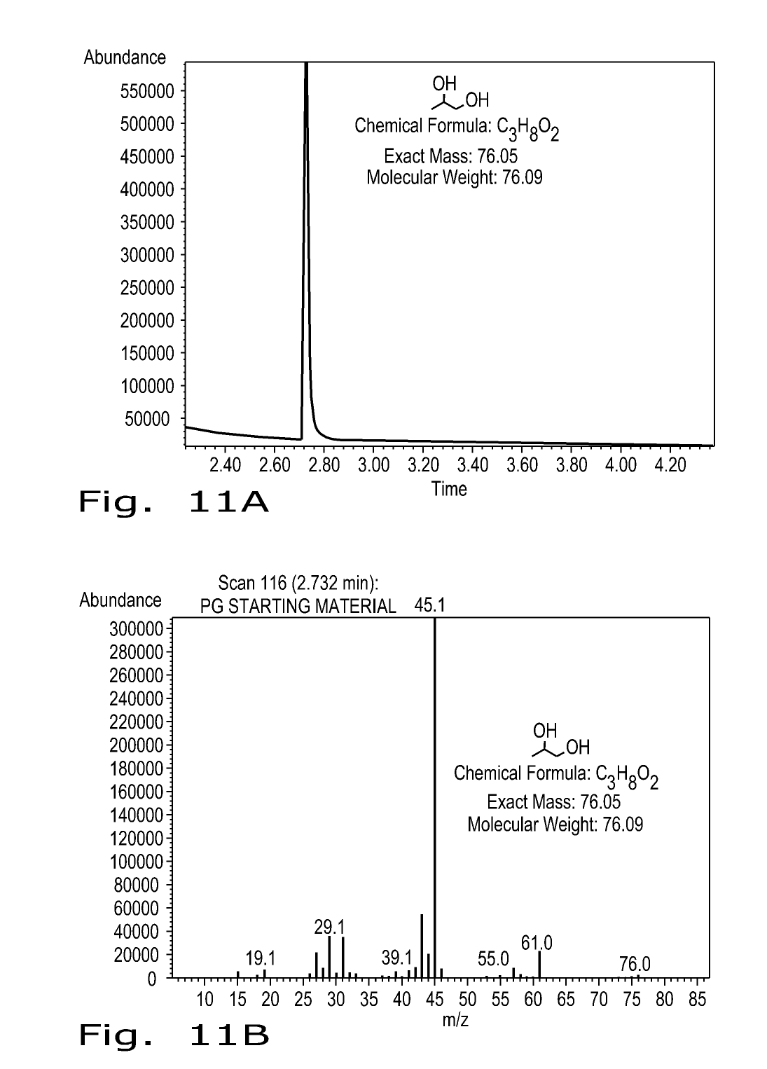

FIGS. 11A and 11B, respectively, are GC chromatogram and mass spectrum of propylene glycol (PG) starting material. Representative of reactions involving PG and EG, Scheme 3 shows PG etherification conducted according to Example 4.

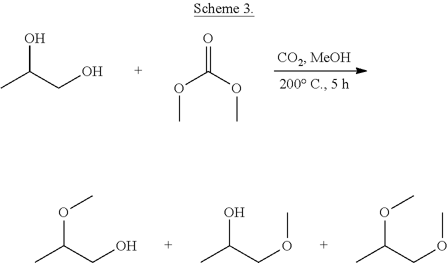

### Example 4

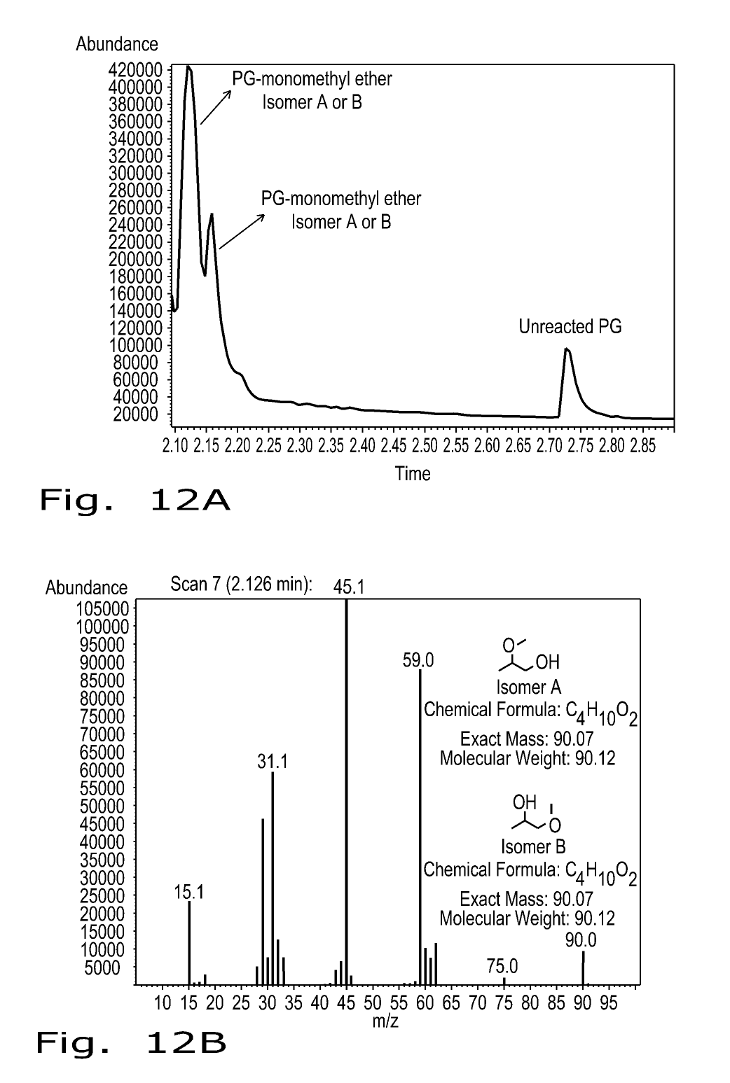

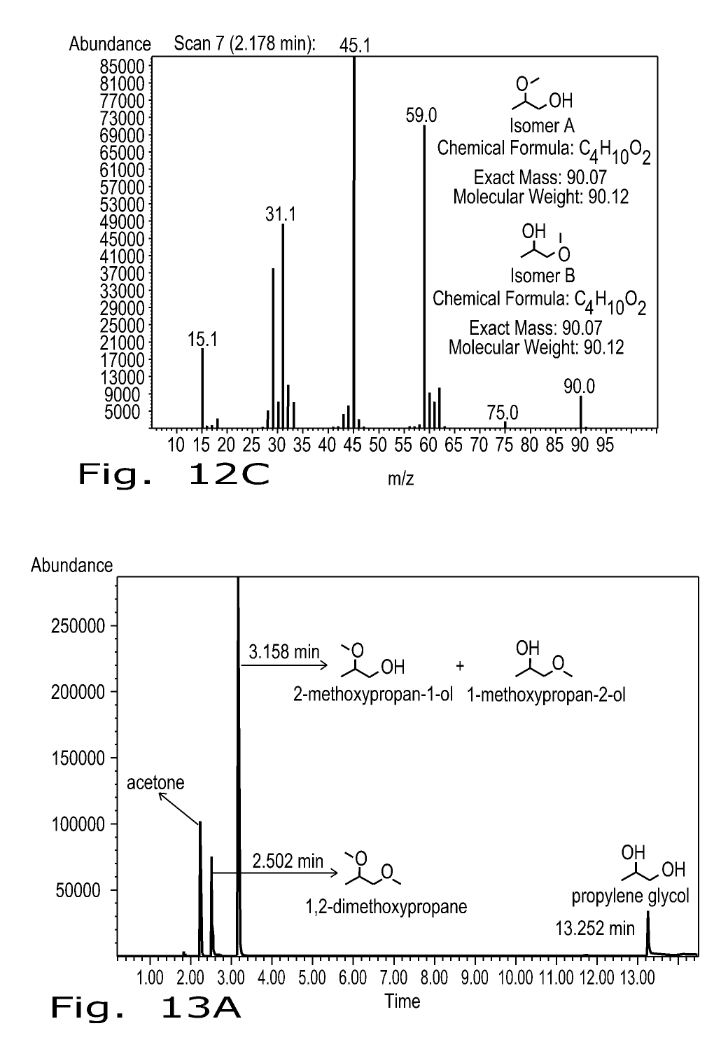

Experimental condition (MeOH, DMC, CO₂). A 250 cc Hastelloy pressure vessel was charged with 10 g of propylene glycol (PG, 131 mmol), 50 g of dimethyl carbonate (DMC, 550 mol, ˜4.2 eq.) and 50 g of MeOH. The vessel was then sealed tightly and affixed to the reactor apparatus, purged x3 with 400 psi of CO₂, then saturated with CO₂ until the pressure remained steady at 400 psi (methanol absorbs considerably amounts of CO₂). While stirring at 700 rpm, the vessel was heated to 200° C., where the reaction persisted for 5 h; the maximum pressure attained was 1890 psi at this temperature. After that time, the solution was cooled to ambient temperature, gas released, and stirring halted. The resultant brownish solution was then analyzed by GC/MS (70° C. initial temp, hold for 4 min, then 10° C. per minute until 300° C., hold for 10 min). The resultant chromatogram (FIG. 12A) revealed a small signal at 2.72 min with m/z of 76.0 (unreacted PG), and two prominent signals at 2.126, 2.159 min both with m/z of 90.0, consistent with the monomethylether isomers of PG. FIGS. 12B and 12C show the mass spectrum of the PG-monoethyl ether isomers A or B.

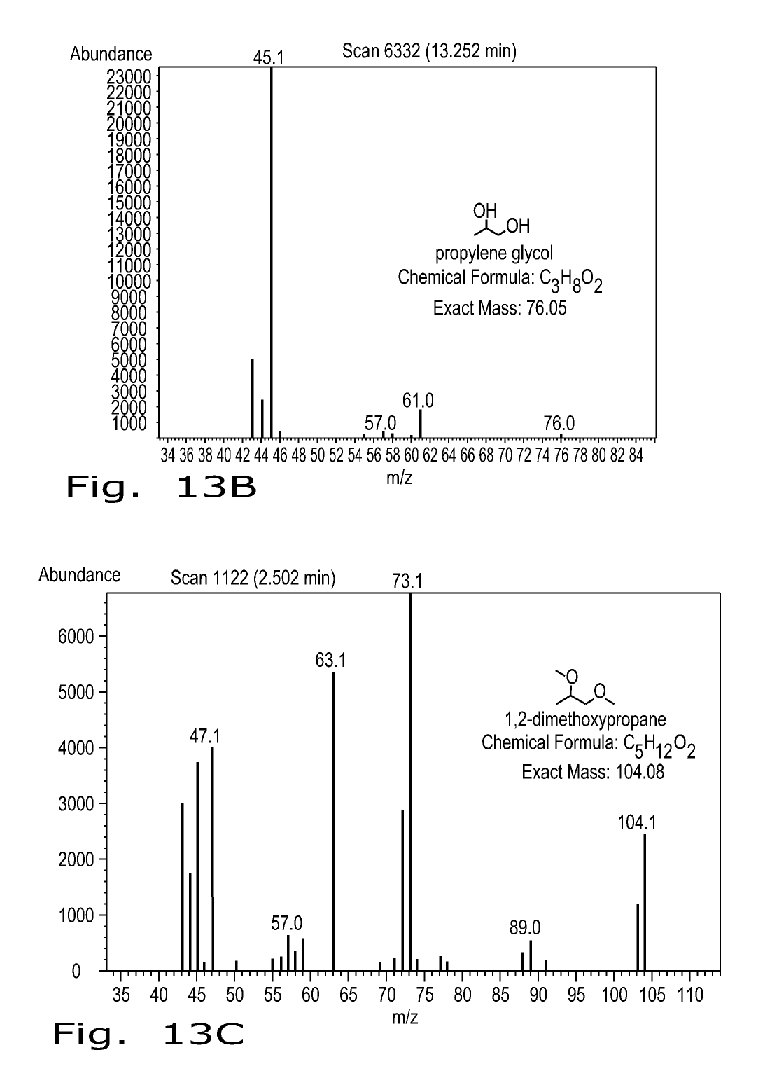

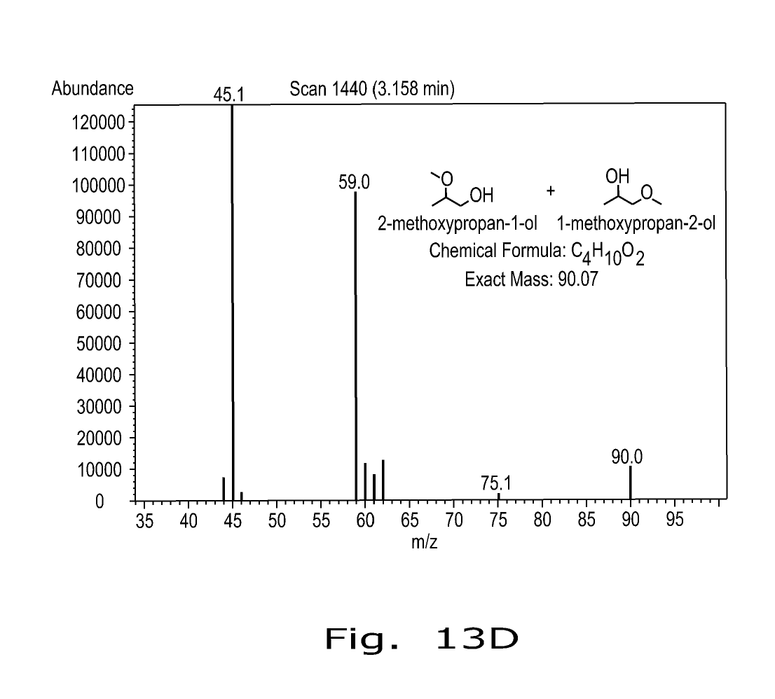

GC/MS analysis using a HP Innowax column and following inlet and oven temperature ramps: Inlet—60° C. initial, hold for 1 min, ramp 5° C. per min until 100° C., no hold, ramp 60° C. per min until 250° C.; Oven—70° C. initial, hold for 5 min, ramp 10° C. per min until 150oC, no hold, ramp 20° C. per minute until 240 min, no hold. The results are presented in FIGS. 13A-13D.

FIG. 13A, is a gas chromatogram of the mixed mono- and di-methyl ether products of PG etherification according to an embodiment of the present process. FIG. 13B is the mass spectrum corresponding to the signal at 13.52 minutes in the gas chromatogram detailed in FIG. 13A, and specifying unreacted propylene glycol. FIG. 13C is the mass spectrum corresponding to the signal at 2.502 minutes in the gas chromatogram detailed in FIG. 13A, and denoting PG dimethyl ether (1,2-dimethoxypropane). FIG. 13D is a mass spectrum corresponding to the signal at 3.158 minutes in the gas chromatogram detailed in FIG. 13A, and represents isomers of PG monomethyl ether (1-methoxypropan-2-ol and 2-methoxypropan-1-ol).

Both the chromatograms and corresponding spectra reveal clearly a high rate of conversion of PG to the preponderant monomethyl ethers, which did not separate, as well as a significant amount of the dimethyl ethers. The control experiment (no CO2) manifested only unreacted PG.

### Comparative Example 7

Experiment condition (No CO₂). A 250 cc Hastelloy pressure vessel was charged with 10 g of propylene glycol (PG, 131 mmol), 50 g of dimethyl carbonate (DMC, 550 mol, ˜4.2 eq.) and 50 g of MeOH. The vessel was then sealed tightly and affixed to the reactor apparatus, and heated to 200° C. with an overhead stirring rate of 700 rpm for 5 h. After that time, the solution was cooled to ambient temperature and stirring halted. The resultant brownish solution was then analyzed by GC/MS (70° C. initial temp, hold for 4 min, then 10° C. per minute until 300° C., hold for 10 min). The resultant chromatogram revealed only a signal at 2.72 min with m/z of 76.0, corresponding to unreacted PG as in FIG. 11A.

### D. Isosorbide Allylation

Representative of sugar alcohols, sorbitol is subject to cyclic dehydration to form isosorbide. In Scheme 4, the isosorbide is converted to corresponding monoallyl stereoisomers.

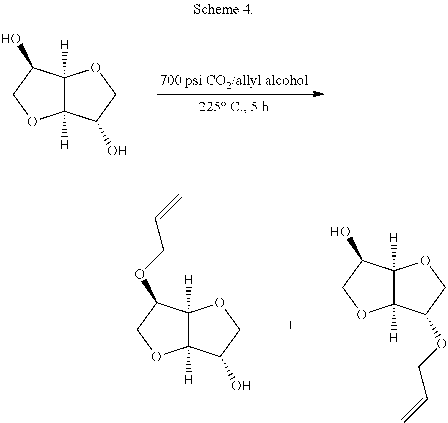

### Example 5

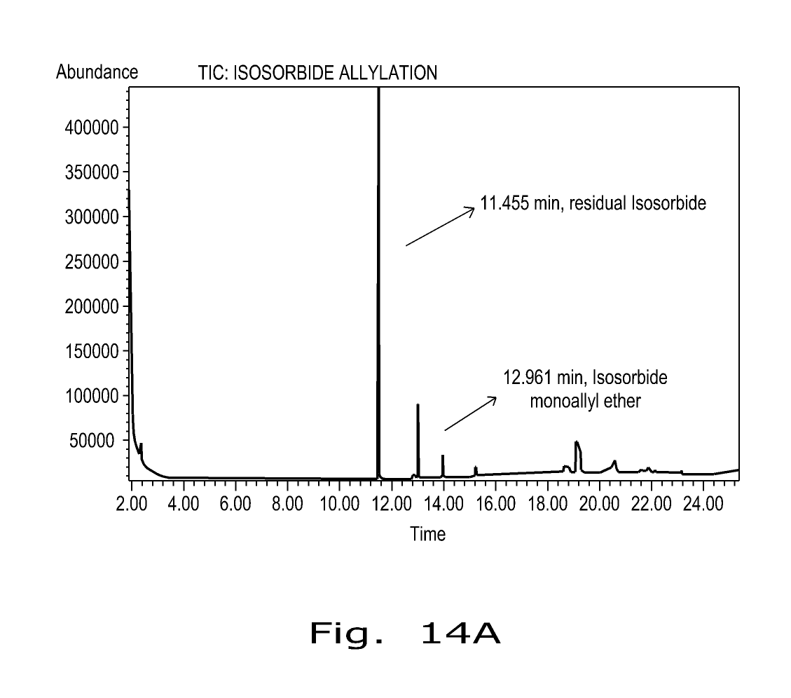

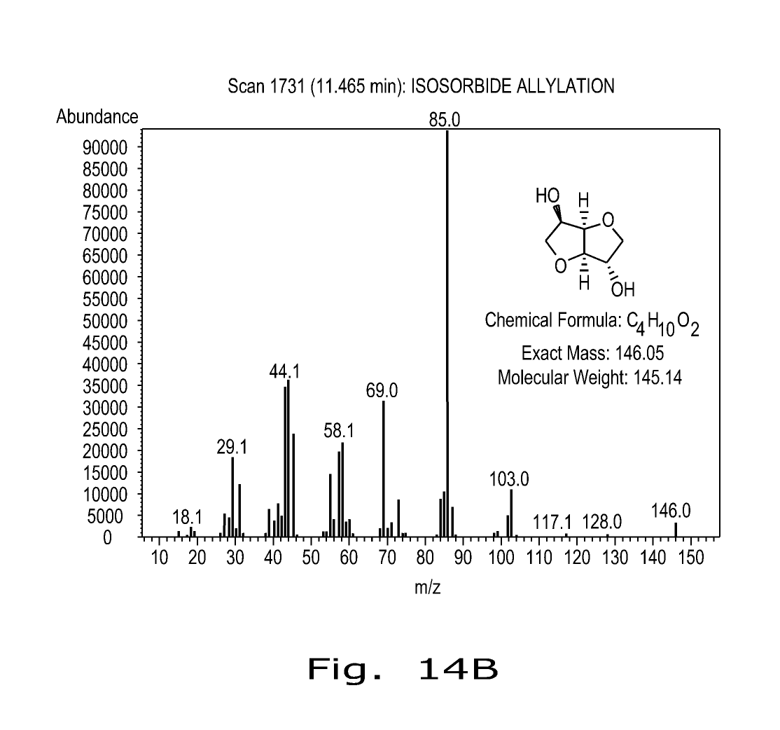

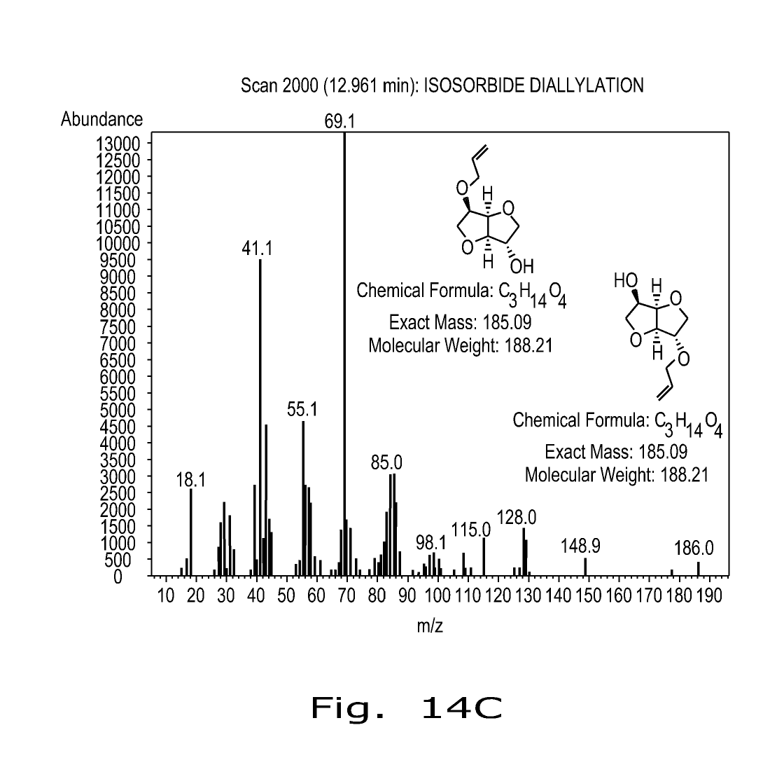

Experimental condition (DMC, CO₂, allyl alcohol). A 300 cc stainless steel pressure vessel was charged with 10 g of isosorbide and 70 g of allyl alcohol. After the vessel was sealed, the head space was purged ×3 with 600 psi of CO₂, then pressurized to 700 psi CO₂. While overhead stirring at 600 rpm, the vessel was heated to 225° C., the temperature at which the reaction proceeded for 5 h. The pressure read 1662 psi at this temperature. After cooling to room temperature followed by depressurization, the products were transferred to a 100 mL glass storage container and contents analyzed by GC/MS. FIG. 14A is a gas chromatogram showing an analysis of isosorbide allylation products according to Example 5. Two peaks represents residual isosorbide at 11.465 min. and isosorbide monoallyl ether at 12.961 min. FIG. 14B shows the mass spectrum of unreacted isosorbide signal, and FIG. 14C shows the mass spectrum of isosorbide monoallyl ether isomers signal.

With process optimization the diether species also can be generated in significant quantities. We envision that this can be a pathway to generate allyl ethers. Allyl ethers then can be subjected to metathesis (polymerization) and/or epoxidation to give a range of versatile derivative compounds. The products from these reactions can be used, for example, in plasticizers, epoxy glue, polycarbonates, or ink toners.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently know or to be developed, which may be used within the scope of the invention. Therefore, unless changes otherwise depart from the scope of the invention, the changes should be construed as being included herein.

We claim:

1. A process for preparing mono- or dialkyl ethers comprising: contacting a diol with an alkylating agent in an alcoholic solvent, in the presence of a catalyst that generates in situ, at a reaction temperature for a sufficient time to convert said diol to a corresponding alkyl ether.

2. The process according to claim 1, wherein said catalyst is hydrated carbon dioxide (CO2) that forms carbonic acid.

3. The process according to claim 2 wherein said weak acid is carbonic acid.

4. The process according to claim 1, wherein said diol is selected from the group consisting of: an isohexide, a reduction product of 5-hydroxymethylfurfural (HMF), ethylene glycol (EG), propylene glycol (PG), 2,3 butane diol (BDO), and 1,6 hexane diol.

5. The process according to claim 4, wherein said isohexide is at least one of: isosorbide, isomannide, and isoidide.

6. The process according to claim 4, wherein said reduction product of HMF includes furan-2,5-diyldimethanol (FDM), ((2R,5S)-tetrahydrofuran-2,5-diyl)dimethanol and ((2S,5S)-tetrahydrofuran-2,5-diyl)dimethanol (bHMTHFs).

7. The process according to claim 1, wherein said alkylating agent is an alkyl carbonate.

8. The process according to claim 7, wherein said alkyl carbonate is selected from the group consisting of: dimethyl carbonate (DMC), diethyl carbonate (DEC), and dipropyl carbonate (DPC).

9. The process according to claim 1, wherein said alcohol solvent is a primary alcohol or an allyl alcohol.

10. The process according to claim 9, wherein said alcohol solvent is selected from the group consisting of methanol, ethanol, propanol, and butanol.

11. The process according to claim 1, wherein said contacting of said diol and alkylating agent is in an atmosphere in which CO2 comprises at least 5%.

12. The process according to claim 11 wherein said atmosphere is ≧50% CO₂.

13. The process according to claim 2, wherein said CO2 is at an initial pressure of at least 100 psi to about 800 psi.

14. The process according to claim 1, wherein said reaction temperature is about 150° C. to about 260° C.

15. The process according to claim 1, wherein said reaction is for a duration of about 3 hours to about 12 hours.

16. The process according to claim 1, wherein said alkyl carbonate is present in excess, at least 2 fold greater than that of the diol.

17. The process according to claim 1, wherein said alcoholic solvent is present in excess, at least 2 fold greater than that of the diol.

18. The process according to claim 1, wherein said diol is converted to said corresponding mono- and dialkyl ethers in yields of greater than 50% of a starting amount of diol.

19. The process according to claim 1, wherein said mono- or dialkyl ether is an allyl ether.

20. A process of making a polyether or epoxide, comprising preparing a mono- or dialkyl ether by contacting a diol with an alkylating agent in an alcoholic solvent, catalyzed by a weak acid generated in situ.

21. The process according to claim 20, wherein said mono- or dialkyl ether is an allyl ether.

22. The process according to claim 21, further comprising at least polymerizing or epoxidizing said allyl ether.

23. The process according to claim 20, wherein said ethers are derived from a diol selected from the group consisting of ethylene glycol (EG), propylene glycol (PG), 2,3 butane diol (BDO), 1,6 hexane diol, isosorbide, isomannide, isoidide, furan-2,5-diyldimethanol (FDM), ((2R,5S)-tetrahydrofuran-2,5-diyl)dimethanol and ((2S,5S)-tetrahydrofuran-2,5-diyl)dimethanol (bHMTHFs).
